(19) United States
(12) Patent Application Publication
(10) Pub. No.: US 2012/0121618 A1
(43) Pub. Date: May 17, 2012

(54) Predicting And Treating Prostate Cancer

(75) Inventors: Philip W. Kantoff, Brookline, MA (US); William K. Oh, Pleasantville, NY (US); June M. Chan, Oakland, CA (US); Wanling Xie, Newton, MA (US); Miyako Abe, Brookline, MA (US); Lorelei A. Mucci, Newton, MA (US)

(73) Assignee: DANA-FARBER CANCER INSTITUTE, INC.

(21) Appl. No.: 13/201,426

(22) PCT Filed: Feb. 12, 2010
(86) PCT No.: PCT/US10/24122

§ 371 Date: Jan. 23, 2012

(30) Foreign Application Priority Data
Feb. 12, 2009 (US) 61152119

(51) Int. Cl. C12Q 1/68; G01N 33/53; A61K 31/573; A61K 45/00; A61K 38/44; B65D 71/00; A61P 39/06; A61P 37/04; H01J 49/26; B01J 19/12; G06Q 50/24; B65D 85/00; C40B 30/10; A61P 35/00

(52) U.S. Cl. 424/184.1; 506/12; 435/6.11; 436/501; 514/179; 424/94.4; 250/282; 250/492.1; 705/3; 206/459.5; 206/232

(57) ABSTRACT

The disclosure features methods and compositions for determining whether a male subject has, or is at an increased risk of developing, an aggressive form of prostate cancer. The disclosure also features methods for adjusting a treatment regimen (e.g., discontinuing a therapy comprising an antioxidant) or administering a therapy that does not contain an antioxidant for a male subject in view of one or both of an MnSOD2 genotype status and an elevated level of an antioxidant in a biological sample obtained from the male subject. Also featured are methods for reducing superoxide levels in a subject based on one or both of an MnSOD2 genotype status and an elevated level of an antioxidant in a biological sample obtained from the male subject.

### STATEMENT REGARDING FEDERALLY SPONSORED RESEARCH OR DEVELOPMENT

The research described in this application was supported in part by grant no. CA106947 from the National Cancer Institute of the National Institutes of Health. Thus, the Government has certain rights in the invention.

### BACKGROUND

Prostate cancer effects more than 200,000 men each year in the United States alone and is second only to lung cancer in the number of deaths. If detected and treated early, a number of treatment options are available to treat and even cure prostate cancer. Therefore, effective methodologies for early diagnosis of, and/or prediction of risk for developing, prostate cancer, especially aggressive prostate cancer, are greatly needed.

### SUMMARY

The disclosure relates to determining whether a male subject (e.g., a man) has, or is at an increased risk for developing, an aggressive form of prostate cancer. The findings described herein demonstrate that men were more likely to develop an aggressive form of prostate cancer if the men had elevated levels of selenium in their blood or elevated levels of selenium and one of several manganese superoxide dismutase 2 (MnSOD2 or SOD2) genotypes. Accordingly, the disclosure features a variety of methods and compositions for determining whether a man has, or is at an increased risk for developing, an aggressive form of prostate cancer.

In addition, as elevated levels of antioxidants in these men were not therapeutically effective, and high levels of antioxidants are detrimental to men (see, e.g., Omenn et al. (1996) J. Natl. Cancer Inst. 88(21):1550-1559), the disclosure also features methods for adjusting a treatment regimen (e.g., discontinuing a therapy comprising an antioxidant or administering a therapy that does not contain an antioxidant) for a male subject in view of one or both of the elevated antioxidant levels and their MnSOD2 genotype status.

More specifically, the present disclosure features a method for determining whether a man has, or is at an increased risk of developing, an aggressive form of prostate cancer. The method includes: providing a man having an elevated level of an antioxidant in a biological sample obtained from the man; and determining the MnSOD2 genotype of the man, the genotype being selected from rs4880 (T/C), rs4880 (T/T), rs2758330 (G/G), rs2758330 (G/T), rs2758332 (A/C), rs2758332 (C/C), rs4523113 (A/A), and rs4523113 (T/T). An MnSOD2 genotype of rs4880 (T/C), rs4880 (T/T), rs2758330 (G/G), rs2758330 (G/T), rs2758332 (A/C), rs2758332 (C/C), rs4523113 (A/A), and rs4523113 (T/T) indicates that the man has, or is at an increased risk of developing, an aggressive form of prostate cancer. The method can further involve determining that the man has an elevated level of the antioxidant in the biological sample obtained from the man.

The disclosure provides another method for determining whether a man has, or is at an increased risk of developing, an aggressive form of prostate cancer. The method includes: providing a man having an MnSOD2 genotype selected from rs4880 (T/C), rs4880 (T/T), rs2758330 (G/G), rs2758330 (G/T), rs2758332 (A/C), rs2758332 (C/C), rs4523113 (A/A), and rs4523113 (T/T); and measuring the level of an antioxidant in a biological sample obtained from the man, an elevated level of the antioxidant in the biological sample as compared to a control level indicating that the man has, or is at an increased risk of developing, an aggressive form of prostate cancer. The method can further involve determining the MnSOD2 genotype of the man.

All the methods of the disclosure can include the following embodiments. Thus, the man can have been identified as having prostate cancer and the antioxidant can be or contain selenium. Moreover, it can be or contain lycopene, α-tocopherol, α-carotene, β-carotene, β-cryptoxanthin, γ-tocopherol, lutein, resveratrol, or retinol. It can be or contain a flavenoid. The MnSOD2 genotype can be rs4880 (T/C), rs4880 (T/T), rs2758330 (G/G), rs2758330 (G/T), rs2758332 (A/C), rs2758332 (C/C), rs4523113 (A/A), or rs4523113 (T/T). The determining can include or be determining whether the man has an MnSOD2 genotype of rs4880 (T/C) or rs4880 (T/T). Alternatively, or in addition, the determining can include or be determining whether the man has an MnSOD2 genotype of rs2758330 (G/G), rs2758330 (G/T), rs2758332 (A/C), rs2758332 (C/C), rs4523113 (A/A), or rs4523113 (T/T). The level of the antioxidant in the biological sample can be at least 1.5 fold (e.g., at least 2 fold) greater than the control level. The biological sample can be, for example, a blood sample. Determining the MnSOD2 genotype can be by, or include, a polymerase chain reaction (PCR), or nucleic acid hybridization. Measuring antioxidant levels in the biological sample can include or be by inductively-coupled plasma mass spectrometry (ICP-MS). The method can include obtaining the biological sample from the man.

In addition, any of these methods can further include the following embodiments. The presence or amount of one or both of prostate serum antigen (PSA) and prostate specific membrane antigen (PSMA) levels in a biological sample obtained from the man can be detected. Moreover, if the man is determined to have, or be at an increased risk for developing, an aggressive form of prostate cancer, a record indicating that the subject has, or is at an increased risk of developing an aggressive form of prostate cancer can be created. The record can be created on a computer-readable medium. Furthermore, if the man is determined to have, or be at an increased risk for developing, an aggressive form of prostate cancer, an anti-cancer therapy for the man can be prescribed. Also, if the man is determined to have, or be at an increased risk for developing, an aggressive form of prostate cancer, an anti-cancer therapy can be administered to the man. The anti-cancer therapy can be one that does not include an antioxidant such as, for example, one that is or contains selenium. The anti-cancer therapy can include one or more of a chemotherapeutic agent, a form of radiation treatment, or an immunotherapeutic agent.

Another aspect of the present disclosure is a method for assessing whether antioxidant therapy should be discontinued. The method includes discontinuing an anti-cancer antioxidant therapy for a man having prostate cancer and undergoing the therapy if the man is identified as having: (i) an elevated level of the antioxidant in a biological sample obtained from the man; and (ii) an MnSOD2 genotype selected from rs4880 (T/C), rs4880 (T/T), rs2758330 (G/G), rs2758330 (G/T), rs2758332 (A/C), rs2758332 (C/C), rs4523113 (A/A), and rs4523113 (T/T). The method can further involve determining the MnSOD2 genotype of the man, and/or determining that the level of the antioxidant is elevated in the biological sample, and/or administering to the subject an anti-cancer therapy (as above) that does not comprise an antioxidant.

Another embodiment of the disclosure is a method for predicting whether a man has, or is at an increased risk of developing, an aggressive form of prostate cancer. The method includes: measuring the level of an antioxidant in a biological sample obtained from a man; determining the MnSOD2 genotype of the man; and predicting that the man has, or is at an increased risk of developing, an aggressive form of prostate cancer if (i) the level of the antioxidant in the biological sample is elevated as compared to a control level and (ii) the man has an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T).

The disclosure includes yet another method for determining whether a man has, or is at an increased risk of developing, an aggressive form of prostate cancer. The method involves: providing a medical profile of a man, the profile including information on the level of an antioxidant in a biological sample obtained from the man and the MnSOD2 genotype of the man; and determining whether the man has, or is at an increased risk of developing, an aggressive form of prostate cancer using the information on the level of the antioxidant and the MnSOD2 genotype. In the method, (i) an elevated level of the antioxidant in the biological sample as compared to a control level and (ii) an MnSOD2 genotype selected from rs4880 (T/C), rs4880 (T/T), rs2758330 (G/G), rs2758330 (G/T), rs2758332 (A/C), rs2758332 (C/C), rs4523113 (A/A), and rs4523113 (T/T) indicates that the man has, or is at an increased risk of developing, an aggressive form of prostate cancer. The method can further include generating the medical profile of the man, the generating involving measuring the level of the antioxidant in the biological sample and determining the MnSOD2 genotype of the man using any of the procedures disclosed herein.

Another feature of the disclosure is a computer-based method for determining whether a man has, or is at an increased risk of developing, an aggressive form of prostate cancer. The method involves: providing the level of an antioxidant in a biological sample from a man and the MnSOD2 genotype of the man; inputting the level of the antioxidant and the MnSOD2 genotype into a computer; and calculating a parameter indicating whether the man has, or is at an increased risk of developing, an aggressive form of prostate cancer using the computer and the input level of the antioxidant and the MnSOD2 genotype. The parameter can evaluate (i) an elevated level of the antioxidant in the biological sample as compared to a control level and (ii) an MnSOD genotype selected from rs4880 (T/C), rs4880 (T/T), rs2758330 (G/G), rs2758330 (G/T), rs2758332 (A/C), rs2758332 (C/C), rs4523113 (A/A), and rs4523113 (T/T) as an indication that the man has, or is at an increased risk of developing, an aggressive form of prostate cancer. The method can further involve outputting the parameter from the computer.

Also provided by the disclosure is a method for prescribing an anti-cancer therapy. The method includes prescribing for a man in need thereof an anti-cancer therapy that does not comprise an antioxidant, the man having been identified as having: (i) prostate cancer; (ii) an MnSOD2 genotype selected from rs4880 (T/C), rs4880 (T/T), rs2758330 (G/G), rs2758330 (G/T), rs2758332 (A/C), rs2758332 (C/C), rs4523113 (A/A), and rs4523113 (T/T); and (iii) an elevated level of the antioxidant in a biological sample obtained from the man as compared to a control level.

In another aspect, the disclosure includes a method for treating prostate cancer. The method includes: administering to a man in need thereof an effective amount of an anti-cancer therapy that does not include an antioxidant, the man having been identified as having: (i) prostate cancer; (ii) an MnSOD2 genotype selected from rs4880 (T/C), rs4880 (T/T), rs2758330 (G/G), rs2758330 (G/T), rs2758332 (A/C), rs2758332 (C/C), rs4523113 (A/A), and rs4523113 (T/T); and (iii) an elevated level of the antioxidant in a biological sample obtained from the man as compared to a control level as described herein.

In yet another method for determining whether a man has, or is at an increased risk of developing, an aggressive form of prostate cancer, the level of an antioxidant in a biological sample obtained from a man is measured. In the method, (i) an elevated level of the antioxidant in the biological sample, as compared to a control level, indicates that the man has, or is at an increased risk of developing, an aggressive form of prostate cancer. The man can have been identified as having prostate cancer. In any aspect of the method, it can further include determining the MnSOD2 genotype of the man and the antioxidant can be or contain selenium.

The disclosure also features an article of manufacture that includes: a container; and a composition contained within the container, the composition containing an active agent for treating a prostate cancer in a man, the active agent in the composition not containing an antioxidant. The container can include a label indicating that the composition is for use in treating prostate cancer in a man if the subject has been identified as having: (i) prostate cancer; (ii) an MnSOD2 genotype selected from rs4880 (T/C), rs4880 (T/T), rs2758330 (G/G), rs2758330 (G/T), rs2758332 (A/C), rs2758332 (C/C), rs4523113 (A/A), and rs4523113 (T/T); and (iii) an elevated level of the antioxidant in a biological sample obtained from the man as compared to a control level. The article of manufacture can further include instructions for administering the composition to a man.

Another provision of the disclosure is a kit for use in determining whether a man is at an increased risk of developing an aggressive form of prostate cancer. The kit can contain: one or more reagents for determining the MnSOD2 genotype of a man; and one or more reagents for measuring the level of an antioxidant in a biological sample obtained from a man. The kit can include instructions for determining whether a man is at an increased risk of developing an aggressive form of prostate cancer and/or means for obtaining a biological sample from a man It can also further include a control (or standard) sample containing a known amount of one or more antioxidants. In the kit, the at least one of the one or more reagents for determining the MnSOD2 genotype of a man can be a nucleic acid primer. Moreover, each of the one or more reagents for determining the MnSOD2 rs4880 genotype of a man can include at least two polynucleotides (e.g., probes) that selectively hybridize to one or more of a nucleic acid sequence containing: rs4880 (C/C), rs4880 (T/C), rs4880 (T/T), rs2758330 (G/G), rs2758330 (G/T), rs2758332 (A/C), rs2758332 (C/C), rs4523113 (A/A), or rs4523113 (T/T). In the kit, the probes can be bound to a solid support. Furthermore, at least one of the one or more reagents for measuring the level of an antioxidant in a biological sample obtained from a man is useful for measuring selenium levels in a biological sample.

Also featured by the disclosure is a method for reducing superoxide levels in a man. The method can involve: providing a man identified as having (i) an elevated level of an antioxidant in a biological sample obtained from the male subject and (ii) an MnSOD2 genotype selected from rs4880 (T/C), rs4880 (T/T), rs2758330 (G/G), rs2758330 (G/T), rs2758332 (A/C), rs2758332 (C/C), rs4523113 (A/A), and rs4523113 (T/T); and administering to the man a compound in an amount effective to stimulate the activity of MnSOD2 to thereby reduce superoxide levels in the man.

Another method for reducing superoxide levels in a man can include: providing a man identified as (i) having an elevated level of an antioxidant in a biological sample obtained from the male subject and (ii) identified as having an MnSOD2 genotype selected from rs4880 (T/C), rs4880 (T/T), rs2758330 (G/G), rs2758330 (G/T), rs2758332 (A/C), rs2758332 (C/C), rs4523113 (A/A), and rs4523113 (T/T); and administering to the man a compound in an amount effective to increase the amount of MnSOD2 in the mitochondria of a cell to thereby reduce superoxide levels in the man. The method can further include: determining that the man has an elevated level of an antioxidant in a biological sample obtained from the man; and/or determining the MnSOD2 genotype of the man. The compound can be one that increases the expression of MnSOD2 to thereby stimulate activity. In any embodiments of the method, the compound can contain a nucleic acid encoding MnSOD2 or a biologically active fragment thereof. The nucleic acid can encode a non-disease-associated form of the MnSOD2 protein or a biologically active fragment thereof. Moreover, the nucleic acid can contain a sequence encoding a mitochondrial targeting signal. The method can further involve predicting whether the man is at an increased risk for developing an aggressive form of prostate cancer and/or administering to the man an anti-cancer therapy.

As used herein, an “aggressive prostate cancer” is a cancer that meets one or more of the following clinical criteria: (a) the cancer has a T score of ≧T2b; (b) patients with the cancer have a prostate-specific antigen (PSA) level>10 ng/mL in blood; or (c) the cancer has a Gleason score≧7. T2b is a primary tumor (T) score indicating that a tumor is in only one side (lobe) of the prostate and is in more than half of that side (lobe). Thus, a T score of ≧T2b would be a cancer that has grown to encompass both sides of the prostate.

T categories for prostate cancers include T0, T1, T2, T3, and T4 and range in advancement with T4 being the most advanced. T1 refers to the presence of a prostate tumor, which is not detectably clinically or with imagining. T1 includes three subcategories: T1a, T1b, and T1c. T1a refers to a tumor that is present in less than 5% of prostate tissue resected; T1b refers to a tumor that is present in greater than 5% of prostate tissue resected; T1c refers to a tumor that was present in a needle biopsy performed due to an elevated serum PSA. T2 refers to a prostate tumor that can be felt (palpated) on examination, but has not spread outside the prostate. T2 includes three subcategories as follows. T2a refers to a tumor that is in half or less than half of one of the prostate gland's two lobes; T2b is described above; and T2c refers to a tumor that is in both lobes of the prostate. T3 refers to a the tumor has spread through the prostatic capsule. T3 can be of one of two subcategories as follows. T3a refers to a tumor that has spread through the capsule on one or both sides and T3b refers to a tumor that has invaded one or both seminal vesicles. T4 refers to a tumor that has invaded tissue or structures surrounding the prostate.

As used herein, a male subject can be any mammalian male. For example, a male subject can be a male rat, hamster, gerbil, mouse, rabbit, guinea pig, cat, dog, goat, sheep, pig, cow, horse, non-human primate (e.g., ape, monkey, gorilla, orangutan, macaque, chimpanzee, or lemur) or a human. In some embodiments, the male subject is one who is known to have prostate cancer prior to undergoing a diagnostic method described herein. For example, a method described herein can be used to determine if a male subject having prostate cancer has, or is at risk of developing, an aggressive form of the prostate cancer. In some embodiments, prior to undergoing any of the methods described herein, the subject is known to have a prostate cancer and/or has been administered a therapy comprising an antioxidant. The subject can have had the therapy administered over a course of one (e.g., two, three, four, five, six, seven, eight, nine, 10, 12, 16, 20, or 24 or more) weeks. The subject can have had the therapy administered for more than one (e.g., two, three, four, five, or six or more) year(s).

As used herein, an “antioxidant” is any molecule capable of slowing or preventing the oxidation of other molecules. Antioxidants can, for example, react with oxygen free-radicals, such as superoxides, to thereby prevent or reduce the ability of the free-radicals to oxidize other biological molecules (e.g., DNA, mRNA, or proteins). Antioxidants can be isolated from natural sources or can be man-made. For example, antioxidants can be isolated from, e.g., citrus fruits, Ginkgo, tea, wine, or dark chocolate. Natural antioxidants include, e.g., hesperidin, quercitrin, rutin, tangeritin, flyoglycosides, kaempferol, catechins, and resveratrol. Antioxidants can include, or consist of, selenium, lycopene, tocopherols (e.g., α-tocopherol), tocotrienols, α-carotene, β-carotene, β-cryptoxanthin, γ-tocopherol, lutein, a polyphenol (e.g., resveratrol), retinol, uric acid, lipoic acid, glutathione, melatonin, or ubiquinol. An antioxidant can be a flavenoid (e.g., luteolin, apigenin, tangeritin, quercetin, kaempferol, myricetin, fisetin, isorhamnetin, pachypodol, rhamnazin, hesperetin, naringenin, eriodictyol, homoeriodictyol, dihydroquercetin, or dihydrokaempferol), an isoflavonoid (e.g., genistein, daidzein, or glycitein), or a neoflavonoid (e.g., catechins (e.g., catechin (C), gallocatechin (GC), catechin 3-gallate (Cg), or gallocatechin 3-gallate (GCg)), epicatechins (e.g., epicatechin (EC), epigallocatechin (EGC), epicatechin 3-gallate (ECg), or epigallocatechin 3-gallate (EGCg)), or anthocyanidins (e.g., cyanidin, delphinidin, malvidin, pelargonidin, peonidin, or petunidin)).

As used herein, an “anti-cancer antioxidant therapy” is a therapy prescribed for, or administered to, a subject for the treatment of a cancer, which therapy includes an antioxidant such as any antioxidant described herein. In some embodiments, the therapy includes, e.g., administration of a single composition containing one or more agents for the treatment of a cancer, wherein at least one of the agents is an antioxidant. In some embodiments, the therapy includes administration of multiple compositions, wherein at least one composition contains a antioxidant. In some embodiments, the therapy includes administration of a composition containing an antioxidant along with a surgery (e.g., a resection of a tumor) or a physical therapy regimen. The anti-cancer antioxidant therapy includes, e.g., administration of a chemotherapeutic agent and an antioxidant. In another example, the anti-cancer antioxidant therapy includes, e.g., administration of a composition containing an antioxidant along with an immunotherapy, chemotherapy, or radiotherapy such as any of those described herein.

Sequence “complementarity,” as used herein, refers to the chemical affinity between specific nitrogenous bases as a result of their hydrogen bonding properties (i.e., the property of two nucleic acid chains having base sequences such that an antiparallel duplex can form where the adenines and uracils (or thymine, in the case of DNA or modified RNA) are apposed to each other, and the guanines and cytosines are apposed to each other). Fully complementary sequences, thus, would be two sequences that have complete one-to-one correspondence (i.e., adenine to uracil or thymine, and guanine to cytosine) of the base sequences when the nucleotide sequences form an antiparallel duplex.

As used herein, a nucleic acid (e.g., a hybridization probe) that “selectively hybridizes” is one that binds a target nucleic acid (e.g., genomic or amplified DNA containing a SNP) with higher affinity or avidity as compared to a random nucleic acid sequence and thus excludes random hybridization. Nucleic acids that selectively hybridize to a target sequence include those having at least 70%, 80%, 85%, 90%, 95%, 97%, 98%, and 99% complementarity with the segment of the target sequence to which it selectively hybridizes.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIGS. 1A and 1B are a pair of graphs depicting the Relative Risk (RR) (and 95% confidence intervals (CI)) for aggressive prostate cancer according to quintiles of individual plasma selenium level and separately for each MnSOD2 genotype (TT, TC, and CC) at rs4880 (FIG. 1A) and quintiles of individual plasma selenium level and with MnSOD2 genotype TT and TC at rs4880 groups combined and the MnSOD2 genotype CC at rs4880 group separately (FIG. 1B).

FIG. 2A is a depiction of the nucleotide sequence of cDNA encoding Variant 1 (the longest isoform) of human MnSOD2 (SEQ ID NO:1). The codon (GTT) containing the rs4880 SNP is underlined and in bold font. The stop codon (taa) is included in SEQ ID NO:1.

FIG. 2B is a depiction of the amino acid sequence (SEQ ID NO:2) of Variant 1 (the longest isoform) of human MnSOD2 encoded by SEQ ID NO:1. The amino acid (valine) encoded by the codon containing the rs4880 SNP is underlined and in bold font.

### DETAILED DESCRIPTION

As discussed above, the disclosure features methods and compositions for determining whether a male subject (e.g., a man) has, or is at an increased risk of developing, an aggressive form of prostate cancer. For example, a subject can be at risk of developing an aggressive form of prostate cancer if the level of an antioxidant is found to be elevated in their blood, or if the level of the antioxidant is elevated and they have an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T).

While in no way limiting or exhaustive, exemplary methods and compositions for use in accordance with the disclosure are provided below.

### APPLICATIONS

Methods for determining whether a male subject (e.g., a man) has, or is at an increased risk of developing, an aggressive form of prostate cancer, can include the steps of determining or predicting whether the male subject (e.g., a man) has, or is at an increased risk of developing, an aggressive form of prostate cancer if: (a) the male subject (e.g., a man) is identified as having an elevated level of an antioxidant in a biological sample obtained from the subject; or (b) (a) and also identified as having an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T).

Methods for Determining a Genotype

The methods described herein can include the step of determining the MnSOD2 genotype of a male subject (e.g., a man). Methods for determining a genotype (e.g., an MnSOD2 genotype) of a subject are known in the art and exemplified in the working Examples. Suitable methods for determining a genotype, in this case detecting the presence of one or more single nucleotide polymorphisms (SNPs) in the MnSOD2 gene, include, e.g., Southern blot (see, e.g., Sambrook et al., Molecular Cloning: A Laboratory Manual Second Edition vol. 1, 2 and 3. Cold Spring Harbor Laboratory Press: Cold Spring Harbor, N.Y., USA, November 1989), real-time PCR analysis (see, e.g., Oliver et al. (2000) J. Mol. Diagnostics 2(4):202-208), nucleic acid hybridization techniques (e.g., microarray analysis, in situ hybridization, Southern blot analysis, or Northern blot analysis), allele-specific PCR (e.g., quantitative allele-specific PCR), pyrosequencing, or DNA sequencing (e.g., enzymatic sequencing or Sanger chemistry sequencing).

To detect the presence of one or more SNPs (e.g., rs4880 (T) or rs4880 (C)) using Southern blot analysis, first, genomic DNA is isolated from a biological sample from a subject (e.g., a man), e.g., using a detergent such as NP40 or sodium dodecyl sulfate in conjunction with proteinase K, followed by extraction of nucleic acid with salt (e.g., sodium chloride), and wash with an alcohol (e.g., ethanol). Regions of DNA containing the SNP of interest can be amplified using PCR. The amplicons can be subjected to gel-electrophoresis to separate the nucleic acids by size, and then transferred to a solid support such as a nitrocellulose membrane. To detect the presence of a SNP in the biological sample, the solid support containing the amplicons can be contacted with a detectably-labeled, complementary oligonucleotide probe that specifically hybridizes to the SNP under appropriate stringency conditions. The binding of the probe to an amplicon indicates the presence of the corresponding SNP in the biological sample.

In another example, the presence of a SNP can also be detected using nucleic acid arrays. For example, genomic DNA isolated from a biological sample can be amplified using PCR as described above. The amplicons can be detectably-labeled during the PCR amplification process (e.g., using one or more detectably labeled dNTPs or detectably labeled primers) or subsequent to the amplification process using a variety of chemical or enzymatic techniques such as nick-translation. (See, e.g., Sambrook et al., supra). Following amplification and labeling, the detectably-labeled amplicons are then contacted to a plurality of hybridization nucleic acid probes specific for (and capable of binding to) a corresponding amplicon. Generally, the probes are bound to a solid support (e.g., a glass or silicon chip) and the position of each probe is predetermined on the solid support. The binding of a detectably-labeled amplicon to a corresponding hybridization probe indicates the presence of the SNP amplified in the biological sample. Suitable conditions and methods for detecting a SNP using nucleic acid arrays are further described in, e.g., Lamy et al. (2006) Nucleic Acids Research 34(14): e100; European Patent Publication No. 1234058; U.S. Publication Nos. 20060008823 and 20030059813; and U.S. Pat. No. 6,410,231; the disclosures of each of which is incorporated by reference in its entirety.

In yet another example, the presence of a SNP can be detected by sequencing. Genomic DNA is isolated from a biological sample obtained from the subject and a region of DNA containing the SNP of interest can be amplified by PCR using a primer pair specific for the region. The sequence of the amplified region (and thus the presence of a SNP in the region) can be determined by any number of chemical or enzymatic sequencing techniques including, e.g., Maxam-Gilbert sequencing, chain-termination enzymatic sequencing, or dye-terminator sequencing. (See, e.g., Kileczawa (2005) “DNA Sequencing: Optimizing the Process and Analysis,” Jones & Bartlett Publishers; pages 204; Griffin et al. (2003) “DNA Sequencing Protocols,” Humana Press; and Alphey (1997) “DNA Sequencing: From Experimental Methods to Bioinfomatics,” Springer, 206 pages).

In some embodiments, the methods can be used to determine whether a male subject has a SNP within a single allele of MnSOD2, e.g., rs4880(C), rs4880(C), rs2758330 (G), rs2758330 (T), rs2758332 (A), rs2758332 (C), rs4523113 (A), or rs4523113 (T). In some embodiments, the methods can be used to determine whether a man has a SNP at two different alleles of MnSOD2, e.g., an MnSOD2 genotype of rs4880 (C/T), rs4880 (T/T), rs2758330 (G/G), rs2758330 (G/T), rs2758332 (A/C), rs2758332 (C/C), rs4523113 (A/A), or rs4523113 (T/T). In some embodiments, the methods can be used to determine whether a man does not have an MnSOD2 rs4880 genotype of rs4880 (C/C).

The detection of one or more of any of the MnSOD2 SNPs described herein can use the nucleic acid sequences of the SNPs themselves, and surrounding sequence, e.g., as hybridization polynucleotide probes or primers (e.g., for amplification or reverse transcription). SNP probes should contain a sequence of sufficient length and complementarity to a corresponding SNP region to specifically hybridize with that SNP region under suitable hybridization conditions. For example, the SNP probes can include at least one (e.g., at least two, at least three, at least four, at least five, at least six, at least seven, at least eight, at least nine, at least 10, at least 11, at least 12, at least 15, at least 20, at least 25, at least 30, at least 35, at least 40, at least 45, at least 50, or 55 or more) nucleotides 5′ or 3′ of the SNP of interest. The polymorphic site of each probe (i.e., the SNP region) is generally flanked on one or both sides by sequence that is common to the different alleles.

Any of the methods for detecting a SNP can, optionally, be performed in formats that allow for rapid preparation, processing, and analysis of multiple samples. This can be, for example, in multi-welled assay plates (e.g., 96 wells or 386 wells) or arrays (e.g., nucleic acid chips). Stock solutions for various reagents can be provided manually or robotically, and subsequent sample preparation (e.g., RT-PCR, labeling), pipetting, diluting, mixing, distribution, washing, incubating (e.g., hybridization), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay.

Methods for Determining an Antioxidant Level in a Biological Sample

The methods described herein can also include the step of determining whether the level of an antioxidant is elevated in a biological sample obtained from a subject. Such methods vary depending on the specific antioxidant and the type of biological sample analyzed. Suitable methods are known in the art and exemplified in the working Examples.

For example, selenium concentrations in biological samples (e.g., blood, urine, or tissue samples) from a subject can be determined using instrumental neutron activation analysis, graphite furnace atomic absorption spectrometry, or inductively-coupled plasma mass spectrometry (ICP-MS). (See, e.g., McKown et al. (1978) J. Radioanalyt. Chem. 43(2):411-20; Stibilj et al. (1996) Microchimica Acta 123(1-4):311-315; Minami et al. (1997) Analytical Sciences 13(3):397-402; and Hinojosa Reyes et al. (2003) J. Anal. At. Spectrom. 18:11-16, the disclosures of each of which with regard to detecting and measuring selenium levels is incorporated by reference in its entirety). Yoshizawa et al. (1998) J. Natl. Cancer Inst. 90(16):1219 describes methods for determining selenium levels in toenails using instrumental neutron activation analysis.

To determine the level of, e.g., vitamin E or vitamin C in blood, the blood can be mixed with solvents such as butylated hydroxytoluene and subsequently assessed using reverse-phase high-performance liquid chromatography (HPLC). (See, e.g., Kökçam et al. (2002) Clinical Biochemistry 35:633-639).

Samples and Sample Collection

Suitable biological samples for the methods described herein include any biological fluid, cell, tissue, or fraction thereof, which includes analyte biomolecules of interest such as nucleic acid (e.g., DNA or an antioxidant). A biological sample can be, for example, a specimen obtained from a male subject or can be derived from such a subject. For example, a sample can be a tissue section obtained by biopsy, or cells that are placed in or adapted to tissue culture. A biological sample can also be a biological fluid such as urine, blood, plasma, serum, saliva, semen, sputum, cerebral spinal fluid, tears, finger or toe nails, or mucus, or such a sample absorbed onto a paper or polymer substrate. A biological sample can be further fractionated, if desired, to a fraction containing particular cell types. For example, a blood sample can be fractionated into serum or into fractions containing particular types of blood cells such as red blood cells or white blood cells (leukocytes). If desired, a sample can be a combination of samples from a man such as a combination of a tissue and a fluid sample.

In some embodiments, any of the methods described herein can include the step of obtaining a biological sample from a male subject (e.g., a man). Any suitable methods for obtaining the biological samples can be employed, although exemplary methods include, e.g., phlebotomy, swab (e.g., buccal swab), or fine needle aspirate biopsy procedure. Non-limiting examples of tissues susceptible to fine needle aspiration include lymph node, lung, thyroid, breast, and liver. Samples can also be collected, e.g., by microdissection (e.g., laser capture microdissection (LCM) or laser microdissection (LMD)), bladder wash, smear (PAP smear), or ductal lavage.

Methods for obtaining and/or storing samples that preserve the activity or integrity of molecules (e.g., nucleic acids or proteins) in the sample are well known to those skilled in the art. For example, a biological sample can be further contacted with one or more additional agents such as appropriate buffers and/or inhibitors, including nuclease, protease and phosphatase inhibitors, which preserve or minimize changes in the molecules (e.g., nucleic acids or proteins) in the sample. Such inhibitors include, for example, chelators such as ethylenediamine tetraacetic acid (EDTA), ethylene glycol bis(P-aminoethyl ether) N,N,N1,N1-tetraacetic acid (EGTA), protease inhibitors such as phenylmethylsulfonyl fluoride (PMSF), aprotinin, leupeptin, antipain and the like, and phosphatase inhibitors such as phosphate, sodium fluoride, vanadate and the like. Appropriate buffers and conditions for isolating molecules are well known to those skilled in the art and can be varied depending, for example, on the type of molecule in the sample to be characterized (see, for example, Ausubel et al. Current Protocols in Molecular Biology (Supplement 47), John Wiley & Sons, New York (1999); Harlow and Lane, Antibodies: A Laboratory Manual (Cold Spring Harbor Laboratory Press (1988); Harlow and Lane, Using Antibodies: A Laboratory Manual, Cold Spring Harbor Press (1999); Tietz Textbook of Clinical Chemistry, 3rd ed. Burtis and Ashwood, eds. W.B. Saunders, Philadelphia, (1999)). A sample also can be processed to eliminate or minimize the presence of interfering substances. For example, a biological sample can be fractionated or purified to remove one or more materials that are not of interest. Methods of fractionating or purifying a biological sample include, but are not limited to, chromatographic methods such as liquid chromatography, ion-exchange chromatography, size-exclusion chromatography, or affinity chromatography.

For use in the methods described herein, a sample can be in a variety of physical states. For example, a sample can be a liquid or solid, can be dissolved or suspended in a liquid, can be in an emulsion or gel, and can be absorbed onto a material (e.g., a piece of paper as in a blood spot).

Exemplary biological samples, methods for obtaining the samples, and purification methods (e.g., DNA purification methods) are exemplified in the accompanying Examples.

Medical Profiles and Risk Determination

The methods and compositions described herein can be used to, e.g., (a) determine whether a male subject (e.g., a human male) has an aggressive form of prostate cancer, (b) predict the risk of a male subject developing an aggressive form of prostate cancer, and/or (c) generate a medical profile for a male subject. The medical profile can include information that indicates, e.g., whether the level of one or more (e.g., one, two, three, four, five, six, seven, eight, nine, 10, 11, or 12 or more) antioxidants are elevated in one or more (e.g., two, three, four, five, six, seven, eight, nine, or 10 or more) biological samples and/or information that indicates an MnSOD2 genotype of the male subject (e.g., an MnSOD2 genotype of rs4880 (C/T), rs4880 (T/T), rs2758330 (G/G), rs2758330 (G/T), rs2758332 (A/C), rs2758332 (C/C), rs4523113 (A/A), or rs4523113 (T/T)). The profile can also include information indicating the presence or amount of one or more (e.g., at least three or more, at least four or more, at least five or more, at least six or more, at least seven or more, at least eight or more, at least nine or more, at least 10 or more, at least 11 or more, at least 12 or more, at least 13 or more, at least 14 or more, at least 15 or more, at least 16 or more, at least 17 or more, at least 18 or more, at least 19 or more, at least 20 or more, at least 21 or more, at least 22 or more, at least 23 or more, or at least 24 or more) additional biomarkers associated with prostate cancer or prostate cancer aggressiveness. For example, the medical profile can contain information regarding the expression level of one or more of: prostate specific antigen (PSA), prostate specific membrane antigen (PSMA), survivin, β-catenin, Bcl-2, and prostate cancer antigen 3 (PCA3). The medical profile can also contain one or more additional SNPs associated with prostate cancer (or prostate cancer aggressiveness).

Such profiles can be made by, e.g., one or both of (i) measuring the level of one or more antioxidants in a biological sample from a male subject (or multiple subjects) and (ii) determining the MnSOD2 genotype of the male subject (or multiple subjects). Suitable methods for performing such steps are described herein. Methods for detecting or measuring mRNA or protein expression level of one or more additional biomarkers include, e.g., western blot or dot blot analysis (for proteins) or northern blot, RT-PCR, or microarray analyses (for mRNA) as described above and in Sambrook et al. (supra). The skilled artisan will be aware of commercially available kits for determining the presence or amount of one or more additional biomarkers such as any of those described herein. For example, PCA3 can be detected in urine using PCA3 ProfileR™ (GenProbe, Inc.) and PSA can be detected in blood using, e.g., BioSafe® PSA4 Prostate Cancer Screening Test (Craig Medical). Methods for detecting survivin in biological fluids are described in, e.g., U.S. Pat. No. 7,097,966.

The resultant information contained within the profile can be used for predicting whether the male subject (e.g., the human male) has, or is at an increased risk for developing, an aggressive form of prostate cancer. In addition, the profiles can be used in a variety of other methods including, e.g., predicting the response of a male subject to a variety of therapies, determining whether the subject has one or more additional medical conditions, whether or not physiologic or behavioral symptoms of the disorder have become apparent. The profiles can also be used to select an appropriate treatment regimen for a male subject identified as having an aggressive form of prostate cancer or at risk of developing an aggressive form of prostate cancer. For example, if a male subject is determined to have an elevated level of an antioxidant in a biological sample obtained from the subject, a medical practitioner can elect to discontinue, or to not prescribe for the subject, any therapy that contains an antioxidant. Where a profile is used to determine that the subject does not have, or is not at risk of developing an aggressive form of prostate cancer, the profile can also include information relevant to a medical practitioner for selecting and/or administering an appropriate treatment regimen. (See below). In some embodiments, the profile can include guidance for discontinuing a treatment regimen.

The methods described herein can involve, e.g., comparing the level of an antioxidant in a test biological sample obtained from a male subject (e.g., a man) to a known or a control level of the particular antioxidant of interest. For example, the level of an antioxidant in a test biological sample can be compared to the corresponding level in a healthy subject, or an average level of the antioxidant in multiple (e.g., two, three, four, five, six, seven, eight, nine, 10, 12, 15, 20, 25, 30, 35, 40, 45, 50, 60, 70, 80, 90, or 100 or more) healthy subjects, of the same species. The control level can also be the level of an antioxidant obtained from a subject, or a group of subjects, of the same species who have prostate cancer, but not an aggressive form of prostate cancer. The control level can be one obtained from a subject, or an average of a group of subjects, who share one or more common characteristics with the test male subject. For example, the control level can be obtained from a male subject, or group of male subjects, of the same race, age, weight or body mass index range, height range, country of origin or country of residence, or medical history.

The control level of an antioxidant can be determined by a variety of methods. For example, histogram analysis can be used in which an entire cohort of patients are graphically presented, wherein a first axis represents the level of an antioxidant and a second axis represents the number of subjects in the cohort whose biological sample contain levels of the antioxidant at a given amount. Determination of the control level of an antioxidant can then be made based on an amount which best distinguishes these separate groups. The control level can be a single number, equally applicable to every subject, or the reference level can vary, according to specific subpopulations (or characteristics of the subpopulation) of male subjects. For example, older subjects can have a different control level than younger subjects for the same antioxidant. In addition, a subject with more advanced disease (e.g., a more aggressive form of prostate cancer) can have a different control level than one with a milder form of the disease.

The comparison between a level of an antioxidant in a test biological sample obtained from a male subject and a control level can also include determining if the level of an antioxidant (e.g., selenium or any of the other antioxidants described herein) falls within a range of values predetermined as predictive of whether or not a subject has, or is at risk of developing, an aggressive form of prostate cancer. In some embodiments, the comparison can be, or include, determining if the level of an antioxidant falls above a predetermined cut-off value. A cut-off value is typically a level of an antioxidant, or ratio of the level of an antioxidant with the level of another antioxidant, above which is considered predictive of whether or not the male subject has, or is at risk of developing, an aggressive form of prostate cancer or is, e.g., cause for a retest.

It is understood that a medical profile described herein can be interpreted as a whole (the level of all antioxidants in the profile and the MnSOD2 genotype of the male subject), in parts (certain collections or groups of antioxidants within the profile), or on an antioxidant-by-antioxidant basis (e.g., in conjunction with the MnSOD2 genotype of the male subject).

Some cut-off values are not absolute in that clinical correlations can still remain significant over a range of values on either side of the cutoff; however, it is possible to select an optimal cut-off value of a level of an antioxidant for a particular sample type. Cut-off values determined for use in the methods described herein can be compared with, e.g., published ranges of antioxidant levels, but can also be individualized to the methodology used and a particular population of male subjects. It is understood that improvements or refinements in optimal cut-off values could be determined depending on the sophistication of statistical methods used and on the number and source of biological samples used to determine reference level values for the different antioxidants. Therefore, established cut-off values can be adjusted up or down on the basis of periodic re-evaluations or changes in methodology or population distribution.

In some embodiments, the level of an antioxidant in a test sample that is predictive is at least 1.5 (e.g., 2, 2.5, 3, 3.5, 4, 4.5, 5, 5.5, 6, 6.5, 7, 8, 9, or 10 or more) times greater than the control level.

The methods described herein can be used to determine whether a male subject is at a 1.5 (e.g., 2, 2.5, 3, 3.5, 4, 4.5, 5, 5.5, 6, 6.5, 7, 7.5, 8, 8.5, 9, 9.5, 10, 11, 12, 13, 14, 15, 20, 25, 30, 35, 40, 45, 50, or 55 or more)-fold increased risk for developing an aggressive form of prostate cancer. The increased risk can be, e.g., more than 5 (e.g., 6, 7, 8, 9, 10, 11, 12, 13, 14, 15, 20, 25, 30, 35, 40, 45, 50, 55, 60, 65, 70, 75, 80, 85, 90, or 95 or greater) % greater chance of developing an aggressive form of prostate cancer. The male subject can be at a 1.5-fold, or more than 5% greater, risk for developing an aggressive prostate cancer as compared to, e.g.: (i) a male subject who does not have an elevated level of one or more antioxidants in a biological sample; or (ii) (i) and a MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), or rs4523113 (T/T).

In some embodiments, after determining that a male subject has, or is at an increased risk for developing, an aggressive form of prostate cancer, or determining that the subject does not have or is not at risk for developing the cancer, a record of that determination can be created. For example, a medical profile can be updated to reflect the determination and/or a separate record can be created by a medical professional. The record can be in written form or stored on a computer or other computer-readable medium. (See below).

In some embodiments, the determining can be performed via a network that links medical professionals (e.g., doctors, nurses, or in some cases lab technicians), subjects, and an intermediary server for the purpose of providing medical determination for the subjects (e.g., risk of developing an aggressive form of prostate cancer). The network can be within a health care institution. The professionals can be connected by a single network or can be connected by different internal networks that can communicate, e.g., using secure and/or proprietary protocols. The external network can be the internet or other well-distributed telecommunications network. Information relating to level of one or more antioxidants in a biological sample from a subject and/or an MnSOD2 genotype of the subject can be provided by a first professional and delivered to the another professional by way of the network. A second medical professional can receive the information, make a determination in accordance with the instant disclosure, and transmit the determination back to the first professional, or in some cases, to a third medical professional. Optionally, the subject can be directly notified. For example, a lab technician can process a biological sample to determine a genotype or the level of an antioxidant in the sample and subsequently relay the information to a first doctor by way of the network. Based on the information, the doctor can make a determination as to whether the subject has, or is at an increased risk of developing, an aggressive form of prostate cancer, and optionally transmit that data to the patient or a second doctor who can then select and/or administer an appropriate therapy to the subject. In some cases, the second doctor may decide to discontinue a current therapy based on the determination.

Alternatively, a computer can evaluate at least one parameter based on the information and make a determination. The determination can be transmitted back to a first medical professional and/or to the subject. The transmission by the computer can also include information useful for selecting and/or administering a therapy to the subject.

As described above, the determination can be stored/recorded in a database and/or transmitted to one or more additional health-care providers or insurers. The results can also be made available, e.g., for analysis by public health professionals and/or epidemiologists.

After determining that a male subject has, or is at an increased risk for developing, an aggressive form of prostate cancer, a medical practitioner can select an appropriate therapeutic regimen for the subject (e.g., an anti-cancer agent with or without an antioxidant). Selecting a therapy for a subject can be, e.g.: (i) writing a prescription for a medicament; (ii) giving (but not necessarily administering) a medicament to a subject (e.g., handing a sample of a prescription medication to a patient while the patient is at the physician's office); (iii) communication (verbal, written (other than a prescription), or electronic (email, post to a secure site)) to the patient of the suggested or recommended therapeutic modality; or (iv) identifying a suitable therapeutic regimen for a subject and disseminating (reporting) the information to other medical personnel, e.g., by way of patient record or updating the medical profile. The latter (iv) can be useful in a case where, e.g., more than one therapeutic agent are to be administered to a patient by different medical practitioners.

It is understood that a medical profile described herein can be in electronic form (e.g., an electronic patient record stored on a computer or other electronic (computer-readable) media such as a DVD, CD, or floppy disk) or written form. The medical profile can also include information for several (e.g., two, three, four, five, 10, 20, 30, 50, or 100 or more) subjects (e.g., human patients). Such multi-subject medical profiles can be used, e.g., in analyses (e.g., statistical analyses) of particular characteristics of subject cohorts.

After predicting that a subject has, or is at an increased risk for developing an aggressive form of prostate cancer, a medical practitioner (e.g., a doctor) can administer the appropriate therapeutic regimen to the subject. Treatment regimens for prostate cancer can include, e.g., surgery (transurethral resection of the prostate, radical prostatectomy, and/or removal of one or more testes), radiation therapy (e.g., external beam therapy using high-energy x-rays or seed-implantation therapy), hormone therapy (e.g., one or more agents that inhibit production of testosterone, e.g., leuprolide and goserelin), cryotherapy, gene therapy, immunotherapy, or chemotherapy. Suitable chemotherapeutic agents for use in treating prostate cancer include, but are not limited to, Taxotere®, Emcyt® (estramustine), mitoxantrone, or prednisone. Methods of administering anti-cancer agents are known in the medical arts.

Methods for Reducing Superoxide Levels in a Subject

The disclosure also features methods for reducing superoxide levels in a male subject (e.g., a man). The method can include administering to a male subject an effective amount of a compound that (a) stimulates the activity of MnSOD2 and/or (b) increases the amount of MnSOD2 in the mitochondria of a cell, to thereby reduce superoxide levels in the male subject. It is understood that in some embodiments, stimulating an increase in expression of MnSOD2 mRNA or protein can also result in one or both of an increase in cellular MnSOD2 activity and an increase in the amount of MnSOD2 in the mitochondria of a cell. Expression can be mRNA expression or protein expression. Methods for determining mRNA or protein expression are described above. The male subject can be one identified as having (i) an elevated level of an antioxidant in a biological sample obtained from the male subject and (ii) an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T). In some embodiments, the methods can include the steps of identifying the subject as having the elevated level of the antioxidant and/or determining the MnSOD2 genotype of the subject (as described herein). The compound can be, e.g., a small molecule, a nucleic acid (e.g., a nucleic acid encoding an MnSOD2 protein; see below), or a protein (e.g., an MnSOD2 protein or a transcription factor that stimulates the expression of the MnSOD2 protein). In some embodiments, the method can include determining if superoxide levels have been reduced by the compound. Methods for detecting superoxide levels in a sample (e.g., a biological sample obtained from a subject) are known in the art and described in, e.g., Anneren et al. (2008) Acta Paediatrica 73(3):345-348; Brosnan et al. (2002) J. Hypertension 20(2):281-286; and Lin et al. (1999) J. Biol. Chem. 274(19):13650-5.

In some embodiments, the compound can include a nucleic acid encoding an MnSOD2 protein or a biologically active fragment thereof. The nucleic acid sequence can include, e.g., a mitochondrial targeting sequence such as, but not limited to, the proline-glutamic acid -X-asparagine (PEXN) (SEQ ID NO:3) motif (wherein X is any amino acid). (See, e.g., Omura (1998) J. Biochem. 123(6):1010-1016; Neve et al. (2001) J. Biol. Chem. 276(14):11317-11322; Yamada et al. (2004) FEBS Letters 578(3):331-6; and Lee et al. (2006) J. Biol. Chem. 281(24):16700-16706). Suitable methods for constructing nucleic acids and expression vectors are well known to those skilled in the art and described in, e.g., Sambrook et al., supra.

A recombinant nucleic acid can be introduced into a cell using a variety of methods, which methods can depend, at least in part, on the type of cell into which the nucleic acid is introduced. For example, delivery of a nucleic acid to animal cells can feature, for example, the introduction of a vector to the cells using calcium phosphate, electroporation, heat shock, liposomes, or transfection reagents such as FUGENE® or LIPOFECTAMINE®, or by contacting naked nucleic acid vectors with the cells in solution (see, e.g., Sambrook et al., supra).

Administration of a compound described herein or pharmaceutical composition thereof can be systemic or local. Pharmaceutical compositions can be formulated such that they are suitable for parenteral and/or non-parenteral administration. Specific administration modalities include subcutaneous, intravenous, intramuscular, intraperitoneal, transdermal, intrathecal, oral, rectal, buccal, topical, nasal, ophthalmic, intra-articular, intra-arterial, sub-arachnoid, bronchial, lymphatic, vaginal, and intra-uterine administration.

Where the compound is a nucleic acid (e.g., a nucleic acid encoding an MnSOD2 protein or biologically active fragment thereof), the compound can be delivered to a cell in a subject by, for example, the use of polymeric, biodegradable microparticle or microcapsule delivery devices known in the art.

Another way to achieve uptake of the nucleic acid by a cell in a subject is using liposomes, prepared by standard methods. The nucleic acid compounds can be incorporated alone into these delivery vehicles or co-incorporated with tissue-specific or tumor-specific antibodies. Alternatively, one can prepare a molecular conjugate composed of a plasmid or other vector attached to poly-L-lysine by electrostatic or covalent forces. (See, e.g., Cristiano et al. (1995), J. Mol. Med. 73:479). Alternatively, tissue specific expression of a delivered nucleic acid compound can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

The frequency of dosing for a compound is within the skills and clinical judgement of medical practitioners (e.g., doctors or nurses). Typically, the administration regime is established by clinical trials which may establish optimal administration parameters. However, the practitioner may vary such administration regimes according to the subject's age, health, weight, sex and medical status. The frequency of dosing can be varied depending on whether the treatment is prophylactic or therapeutic.

As defined herein, a “therapeutically effective amount” of a compound is an amount of the compound that is capable of producing a medically desirable result (e.g., amelioration of one or more symptoms of a prostate cancer) in a treated subject. A therapeutically effective amount of a compound (i.e., an effective dosage) includes milligram, microgram, nanogram, or picogram amounts of the reagent per kilogram of subject or sample weight (e.g., about 1 nanogram per kilogram to about 500 micrograms per kilogram, about 1 microgram per kilogram to about 500 milligrams per kilogram, about 100 micrograms per kilogram to about 5 milligrams per kilogram, or about 1 microgram per kilogram to about 50 micrograms per kilogram).

The male subject can be any of those described herein, e.g., a man

### Arrays and Kits

Nucleic acid arrays and kits including the arrays are useful in, e.g., detecting the presence of one or more SNPs. The kits and compositions are also useful for determining whether a male subject has, or is at an increased risk for developing, an aggressive form of prostate cancer and/or can be used in conjunction with any of the methods described herein.

The nucleic acid arrays can include at least two (e.g., at least three, at least four, at least five, at least six, at least seven, at least eight, at least nine, at least 10, at least 11, at least 12, at least 15, at least 20, or at least 22) polynucleotides that hybridize to each of at least two (e.g., at least three, at least four, or at least five) SNPs (MnSOD2 genotypes) selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T). The polynucleotide can include sequence of the sense strand or the anti-sense strand of the coding sequence of the MnSOD2 gene containing any of the above SNPs.

The polynucleotide can also be single or double-stranded and of variable length. In some embodiments, the length of one strand of a polynucleotide that hybridizes to a nucleotide sequence including a SNP (e.g., of rs4880 (C), rs4880 (T), rs2758330 (G), rs2758330 (T), rs2758332 (A), rs2758332 (C), rs4523113 (A), or rs4523113 (T)) can be about six nucleotides (e.g., about seven nucleotides, about eight nucleotides, about nine nucleotides, about 10 nucleotides, about 12 nucleotides, about 13 nucleotides, about 14 nucleotides, about 15 nucleotides, about 20 nucleotides, about 25 nucleotides, about 30 nucleotides, about 35 nucleotides, about 40 nucleotides, about 50 nucleotides, about 75 nucleotides, about 100 nucleotides, or about 150 or more nucleotides) in length. A longer polynucleotide often allows for higher stringency hybridization and wash conditions. The polynucleotide can be DNA, RNA, modified DNA or RNA, or a hybrid, where the nucleic acid contains any combination of deoxyribo- and ribo-nucleotides, and any combination of uracil, adenine, thymine, cytosine and guanine, as well as other bases such as inosine, xanthine, and hypoxanthine.

The polynucleotide arrays can be attached to a solid support, e.g., a porous or non-porous material that is insoluble. The substrate can be associated with the support in variety of ways, e.g., covalently or non-covalently bound.

A support can be composed of a natural or synthetic material, an organic material, or inorganic material. The composition of the solid support on which the polynucleotide sequences are attached (either 5′ or 3′ terminal attachment) generally depend on the method of attachment (e.g., covalent attachment). Suitable solid supports include, but are not limited to, plastics, resins, polysaccharides, silica or silica-based materials, functionalized glass, modified silicon, carbon, metals, inorganic glasses, membranes, nylon, natural fibers such as silk, wool and cotton, or polymers. The material comprising the solid support can have reactive groups such as carboxy, amino, or hydroxyl groups, which are used for attachment of the polynucleotides. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, or polymethylpentene (see, e.g., U.S. Pat. No. 5,427,779, the disclosure of which is incorporated by reference in its entirety). Alternatively, the polynucleotide sequences can be attached to the solid support without the use of such functional groups.

Each polynucleotide (of a plurality of polynucleotides) on an array can be immobilized at predetermined positions such that each polynucleotide can be identified by its position. Exemplary polynucleotide arrays for use in the methods and kits described herein are described in, e.g., U.S. Pat. Nos. 6,197,599; 5,902,723; and 5,871,928; the disclosures of each of which are incorporated herein by reference in their entirety.

In some embodiments of any of the arrays described herein, the array of polynucleotides can have less than 100,000 (e.g., less than 90,000; less than 80,000; less than 70,000; less than 60,000; less than 50,000; less than 40,000; less than 30,000; less than 20,000; less than 15,000; less than 10,000; less than 5,000; less than 4,000; less than 3,000; less than 2,000; less than 1,500; less than 1,000; less than 750; less than 500, less than 200, less than 100, or less than 50) different polynucleotides.

The polynucleotide arrays can also be conjugated to solid support particles. Many suitable solid support particles are known in the art and illustratively include, e.g., particles, such as Luminex®-type encoded particles, magnetic particles, and glass particles.

Exemplary particles that can be used can have a variety of sizes and physical properties. Particles can be selected to have a variety of properties useful for particular experimental formats. For example, particles can be selected that remain suspended in a solution of desired viscosity or to readily precipitate in a solution of desired viscosity. Particles can be selected for ease of separation from sample constituents, for example, by including purification tags for separation with a suitable tag-binding material, paramagnetic properties for magnetic separation, and the like.

In some embodiments, encoded particles are used. Each particle includes a unique code (such as a bar code, luminescence code, fluorescence code, a nucleic acid code, and the like). Encoding can be used to provide particles for evaluating different nucleic acids in a single biological sample. The code is embedded (for example, within the interior of the particle) or otherwise attached to the particle in a manner that is stable through hybridization and analysis. The code can be provided by any detectable means, such as by holographic encoding, by a fluorescence property, color, shape, size, weight, light emission, quantum dot emission and the like to identify particle and thus the capture probes immobilized thereto. Encoding can also be the ratio of two or more dyes in one particle that is different than the ratio present in another particle. For example, the particles may be encoded using optical, chemical, physical, or electronic tags. Examples of such coding technologies are optical bar codes fluorescent dyes, or other means. In some embodiments, the particle code is a nucleic acid, e.g., a single stranded nucleic acid.

Different encoded particles can be used to detect or measure multiple nucleic acids (e.g., SNPs) in parallel, so long as the encoding can be used to identify the polynucleotide (corresponding to an analyte nucleic acid) on a particular particle, and hence the presence or amount of the analyte nucleic acid (e.g., a SNP) being evaluated. A sample can be contacted with a plurality of such coded particles. When the particles are evaluated, e.g., using a fluorescent scanner, the particle code, as well as the signal from the detectable label of the probe used to evaluate binding to the polynucleotide associated with the particles, is read.

One exemplary platform utilizes mixtures of fluorescent dyes impregnated into polymer particles as the means to identify each member of a particle set to which a specific capture probe has been immobilized. Another exemplary platform uses holographic barcodes to identify cylindrical glass particles. For example, Chandler et al. (U.S. Pat. No. 5,981,180) describes a particle-based system in which different particle types are encoded by mixtures of various proportions of two or more fluorescent dyes impregnated into polymer particles. Soini (U.S. Pat. No. 5,028,545) describes a particle-based multiplexed assay system that employs time-resolved fluorescence for particle identification. Fulwyler (U.S. Pat. No. 4,499,052) describes an exemplary method for using particle distinguished by color and/or size. U.S. Publication Nos. 2004-0179267, 2004-0132205, 2004-0130786, 2004-0130761, 2004-0126875, 2004-0125424, and 2004-0075907 describe exemplary particles encoded by holographic barcodes.

U.S. Pat. No. 6,916,661 describes polymeric microparticles that are associated with nanoparticles that have dyes that provide a code for the particles. The polymeric microparticles can have a diameter of less than one millimeter, e.g., a size ranging from about 0.1 to about 1,000 micrometers in diameter, e.g., 3-25 μm or about 6-12 μm. The nanoparticles can have, e.g., a diameter from about 1 nanometer (nm) to about 100,000 nm in diameter, e.g., about 10-1,000 nm or 200-500 nm.

Also provided are kits containing any of the nucleic acid arrays described herein. The kits can, optionally, contain instructions for detecting one or more SNPs (e.g., one or more SNPs described herein).

Also provided are kits containing one or more reagents for determining the MnSOD2 genotype of a man (such as any of the arrays described herein); and one or more reagents for measuring the level of an antioxidant in a biological sample obtained from a man The kits can also optionally include instructions for determining if a man has, or is at an increased risk of developing, an aggressive form of prostate cancer based on the level of an antioxidant in a biological sample obtained from the man or the level of the antioxidant in conjunction with the MnSOD2 genotype of the subject. At least one of the one or more reagents for determining the MnSOD2 genotype of a man can be a nucleic acid primer (e.g., a set of primers for amplifying a DNA region of interest containing a SNP).

In some embodiments, the kits can include a means for obtaining a biological sample from a man (e.g., a swab, a lance, or a syringe). The kits can also contain, e.g., a control sample (e.g., a control biological sample) containing a known amount of one or more antioxidants or DNA of a certain MnSOD2 genotype.

The kits can optionally include, e.g., a control labeled-amplicon set containing known amounts of one or more amplicons recognized by nucleic acid probes of an array, if included in the kits. In some instances, the kits can also include an insert (e.g., a paper insert or electronic medium such as a CD, DVD, or floppy disk) reciting control levels, ranges, or thresholds of one or more antioxidants in a biological sample. For example, the insert can recite acceptable levels of antioxidants in a variety of tissue types such as blood or urine.

In some embodiments, the kits can include one or more reagents for processing a biological sample. For example, a kit can include reagents for isolating genomic DNA from a biological sample and/or reagents for amplifying genomic DNA (e.g., a primer set, dNTPs, or a polymerase enzyme). The kits can also, optionally, contain one or more reagents for detectably-labeling genomic DNA or a DNA amplicon, which include, e.g., an enzyme such as a Klenow fragment of DNA polymerase, T4 polynucleotide kinase, one or more detectably-labeled dNTPs, or detectably-labeled gamma phosphate ATP (e.g., ³³P-ATP).

The following are examples of the practice of the invention. They are not to be construed as limiting the scope of the invention in any way.

### EXAMPLES

### Example 1

### Materials and Methods

Study Population. The study population was a cohort of prostate cancer survivors with banked biospecimens in the Prostate Clinical Research Information System (Prostate CRIS) at the Harvard/Dana-Farber Cancer Institute. To be eligible for this retrospective cohort, patients had to have: (a) a diagnosis of localized/regional prostate cancer (e.g., T3 or less, N0 and M0) between T1c and T3; (b) consented to donate blood and be followed clinically for research purposes; and (c) donated blood within six months of prostate cancer diagnosis and before any type of therapy. 778 patients who fulfilled these study criteria were identified. Of these, there was sufficient DNA available for analysis among 764 men, and sufficient plasma for selenium analysis for 499 men. A total of 489 patients had complete clinical, selenium and genotype data and were included in this analysis.

Genomic DNA. Genomic DNA was isolated from peripheral blood using a “QIAamp DNA Blood mini kit” (QIAGEN Inc, Valencia, Calif.). DNA concentration was determined using PicoGreen® dsDNA quantitation reagent (Invitrogen, Carlsbad, Calif.). After determining the concentration of isolated DNA, its concentration was adjusted to 5 ng/μl in TE (Tris-EDTA). DNA samples were then subjected to genotyping analysis as described below.

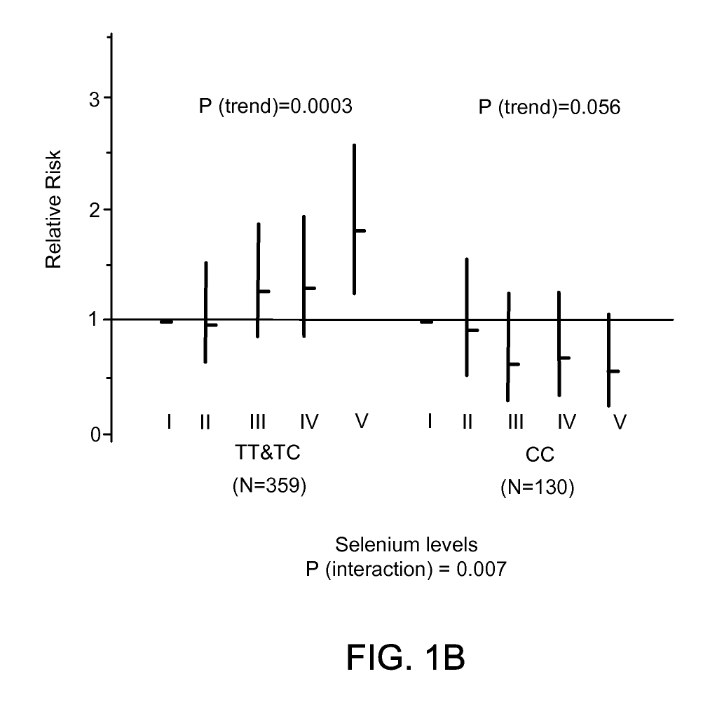

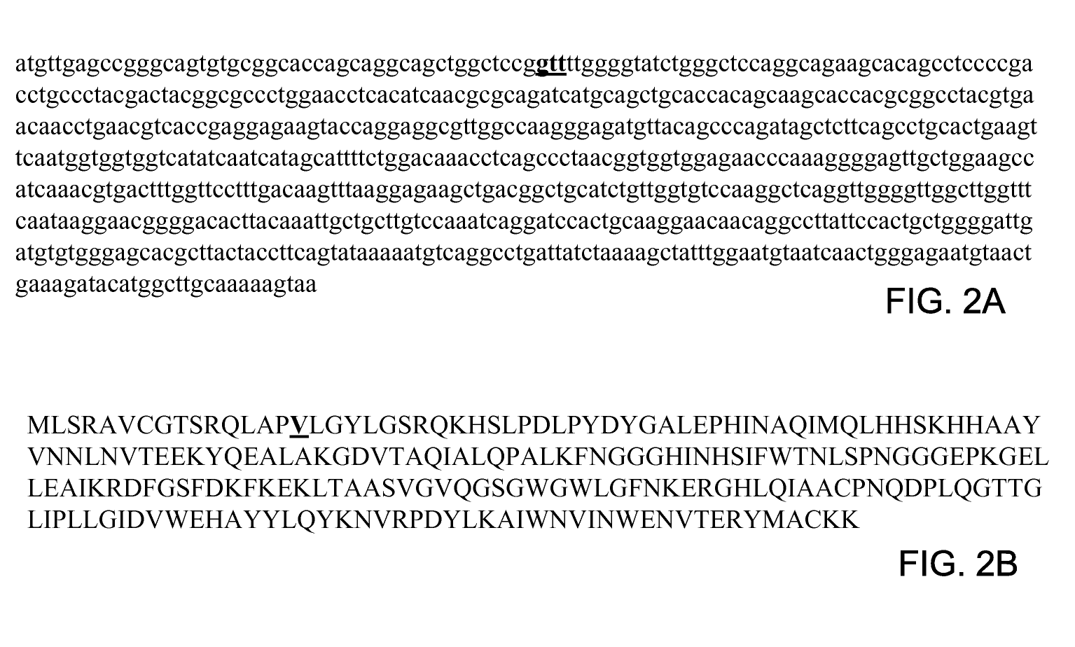

Genotyping. Genotyping all of the SNPs described herein (e.g., the rs4880 (C or T) on the superoxide dismutase 2 (SOD2 or MnSOD2) gene) was performed by iPEX Assay (Increased Plexing Efficiency and Flexibility for MassARRAY System) through single base primer extension with mass-modified terminators (Sequenom, San Diego, Calif.). The rs4880 polymorphism is located on exon 1 of the MnSOD2 gene. The C(GCT) type polymorphism gives rise to a protein encoding Alanine at position 16, and T(GTT) type polymorphism encodes a Valine at position 16. The codon (GTT) containing the rs4880 (C or T) SNP is bolded and underlined in the cDNA sequence (SEQ ID NO:1) encoding variant 1 (the longest isoform) of the human MnSOD2 gene (Genbank Accession No: NM_{—}000636) (FIG. 2A). The NCBI Entrez database entry describing NM_{—}000636 is incorporated herein by reference in its entirety. It will be appreciated that in SEQ ID NO:1 the rs4880 SNP nucleotide is T. In the amino acid sequence (SEQ ID NO:2) encoded by SEQ ID NO:1, the amino acid encoded by the rs4880 SNP-containing codon is Valine (bolded and underlined in FIG. 2B) As part of internal quality control analysis, 47 of the total 764 samples were analyzed twice with blind numbering. Blinded analysis resulted in identical genotypes for all these duplicated samples.

Plasma Selenium Assessment. Blood was collected from patients as part of an ongoing research biospecimen banking program at the Harvard/Dana-Faber Cancer Institute. Plasma was fractionated from the blood and stored at −80° C. until testing. The plasma was subjected to analysis for selenium concentration. Briefly, selenium concentration was assessed using the method described by Satia et al. (2006) Ann. Epidemiol. 16(1):53-58, the disclosure of which is incorporated herein by reference in its entirety.

Statistical methods. Patient disease characteristics at diagnosis were summarized as the number and percentage of patients or as the median, range and interquartile range of levels. MnSOD2 genotype frequencies at rs4880 were summarized as percent with AA, AG, or GG alleles. Plasma selenium levels were categorized to five ordered groups according to quintile cut-off values based on the entire sample (108.3, 118, 125.5, and 139.8 μg/L, respectively).

The primary outcome of interest was presentation of aggressive prostate cancer at diagnosis. Aggressive disease was stage defined as ≧T2b, blood prostate-specific antigen (PSA) level>10 ng/mL, or Gleason score≧7 (corresponding to D'Amico intermediate/high risk categories). For further details on these pathologic scores/categories see D'Amico et al. (1999) J. Clin. Oncol. 17(1): 168-172, the disclosure of which is incorporated herein by reference in its entirety. D'Amico risk categories are as follows: Low Risk (≦T2a and blood PSA≦10 ng/mL and Gleason Score≦6); Intermediate Risk (T2b or PSA 10-20, or Gleason Score 7); and High Risk (>T2b and blood PSA>20 ng/mL and Gleason Score>7).

The Gleason score alone was also considered as an indicator of disease aggressiveness. Association of disease aggressiveness with MnSOD2 genotypes and selenium levels was evaluated using Chi-square test or the Cochran-Armitage test for trend. Relative risk (RR) and 95% confidence intervals (CI) were estimated using a generalized linear model (GLM) for binomial data with a log link rather than a logit link function. Likelihood ratio test from the GLM model was used to test for interaction between rs4880 genotypes and selenium levels on disease aggressiveness, where selenium levels were evaluated both as categorical groups and continuous values.

### Example 2

Demographic and clinical characteristics of this cohort of prostate cancer survivors are provided in Table 1. The median age was 62 years and the median PSA level was 6.0 ng/mL in this predominantly white population. More than half the cohort had low risk disease while about one third intermediate risk and the remainder poor (high) risk.

TABLE 1
Baseline characteristics of 489 men with localized/regional prostate cancer
at diagnosis

| | Median | Range |
|---|---|---|
| Age at diagnosis, years | 62 | 43-86 |
| Interquartile range | | 57-68 |
| PSA at diagnosis, ng/mL | 6.0 | 0.7-575.8 |
| Interquartile range | | 4.7-8.3 |
| Plasma selenium, μg/L | 121.4 | 64.2-221.1 |
| Interquartile range | | 110.4-135.1 |

| | N | % |
|---|---|---|
| Ethnic group | | |
| White | 468 | 95.7 |
| Other | 18 | 3.6 |
| Unknown | 3 | 0.6 |
| T stage at Diagnosis | | |
| T1c | 259 | 53 |
| T2 | 21 | 4.3 |
| T2a | 94 | 19.2 |
| T2b | 9 | 1.8 |
| T3 | 2 | 0.4 |
| T3a | 1 | 0.2 |
| Tx | 103 | 21.1 |
| Biopsy Gleason Score | | |
| 6 or less | 312 | 63.8 |
| 7 | 153 | 31.3 |
| 8 or more | 24 | 4.9 |
| % of biopsy core | | |
| positive* | | |
| ≦33% | 230 | 47 |
| 33%-50% | 59 | 12.1 |
| >50% | 109 | 22.3 |
| Unknown | 91 | 18.6 |
| Disease risk categories** | | |
| High | 46 | 9.4 |
| Intermediate | 167 | 34.2 |
| Low | 276 | 56.4 |
| PSA at diagnosis (ng/ml) | | |
| ≦10 | 405 | 82.8 |
| 10-20 | 52 | 10.6 |
| >20 | 27 | 5.5 |
| Unknown | 5 | 1.0 |

*For details on biopsy core positive scoring, see, e.g., D′Amico et al. (2000) J. Clin. Oncol. 18(6): 1164-1172.
**D′Amico Risk Categories (see above).

The median selenium level was similar among men with different MnSOD2 rs4880 genotypes (≈121 ug/L, p-value=0.38), and MnSOD2 rs4880 genotype alone was not associated with risk of presenting with aggressive prostate cancer (Table 2).

TABLE 2
Summary of MnSOD2 genotypes at rs4880 and their association with plasma selenium
levels and aggressive prostate cancer

| | | Relative risk for aggressive | | | |
|---|---|---|---|---|---|
| | | disease | | Selenium levels (μg/L) | |
| Genotype | N (%) | RR (95%) | P-value* | Median, range | P-value** |
| TT | 107 (21.9) | 1.00 (reference) | 0.346 | 120.9 (85.4~221.1) | 0.376 |
| TC | 252 (51.5) | 1.21 (0.92~1.59) | | 121.5 (64.2~211.6) | |
| CC | 130 (26.6) | 1.10 (0.81~1.51) | | 121.7 (84.2~193.5) | |

*Chi-Square Test
**Kruskal-Wallis test

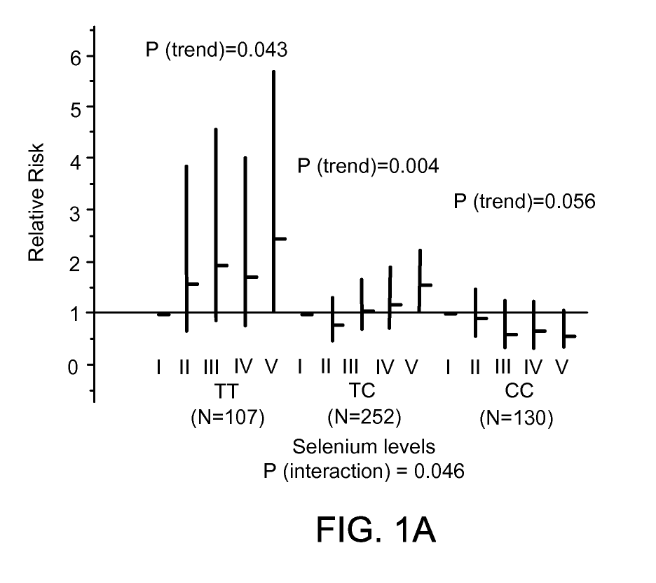

Plasma selenium level was positively associated with risk of aggressive prostate cancer when analyzed alone (p-value=0.04). This effect was statistically significantly modified by MnSOD2 genotype. Among men with the CC allele, those in the highest vs. lowest quintile of plasma selenium had a 40% lower risk of aggressive prostate cancer, although this result was borderline statistically significant (RR=0.60, 95% CI 0.32-1.12; p-value for trend=0.06). Among men with the TT allele, those with high vs. low plasma selenium had an increased risk of presenting with aggressive prostate cancer (RR=2.48, 95% CI 1.07-5.71) (FIG. 1A and Table 3).

TABLE 3
Relative risk (RR) and 95% CI for aggressive prostate cancer according to
quintiles of plasma selenium for 489 men with prostate cancer and stratified
by rs4880 genotype

| | No. of | Quintile levels of plasma selenium | | | | | P*** | P |
|---|---|---|---|---|---|---|---|---|
| rs4880 | patients | I(low) | II | III | IV | V(high) | (trend) | (interaction) |
| TT | 107 | 1.00 | 1.58 | 1.95 | 1.73 | 2.48 | 0.043 | 0.046* |
| | | (reference) | (0.64~3.85) | (0.83~4.58) | (0.74~4.06) | (1.07~5.71) | | (0.018**) |
| TC | 252 | 1.00 | 0.80 | 1.09 | 1.20 | 1.59 | 0.004 | |
| | | (reference) | (0.47~1.35) | (0.70~1.71) | (0.77~1.87) | (1.07~2.35) | | |
| CC | 130 | 1.00 | 0.95 | 0.65 | 0.71 | 0.60 | 0.056 | |
| | | (reference) | (0.57~1.59) | (0.32~1.30) | (0.39~1.31) | (0.32~1.12) | | |

*From the GLM model where selenium was coded as quintile levels;
**From the GLM model where selenium was coded as continuous values;
***Cochran-Armitage test for trend.

TABLE 4
The same date shown in Table 3 but with TT and TC genotypes combined.

| | No. of | Quintile levels of plasma selenium | | | | | P*** | P |
|---|---|---|---|---|---|---|---|---|
| | patients | I(low) | II | III | IV | V(high) | (trend) | (interaction) |
| TT/ | 359 | 1.00 | 0.98 | 1.29 | 1.32 | 1.82 | 0.0003 | 0.007* |
| TC | | (reference) | (0.62~1.53) | (0.87~1.92) | (0.89~1.97) | (1.27~2.61) | | (0.008**) |
| CC | 130 | 1.00 | 0.95 | 0.65 | 0.71 | 0.60 | 0.056 | |
| | | (reference) | (0.57~1.59) | (0.32~1.30) | (0.39~1.31) | (0.32~1.12) | | |

*From the GLM model where selenium was coded as quintile levels;
**From the GLM model where selenium was coded as continuous values;
***Cochran-Armitage test for trend.

There was also a positive but weaker association between selenium and risk of aggressive prostate cancer among those with the TC allele (Tables 3 and 4 and FIG. 1A). Taken together, there was an overall statistically significant interaction between MnSOD2 genotype and plasma selenium level and risk of aggressive prostate cancer (p-value=0.007 when contrasting CC vs. TT/TC and quintiles of selenium) (FIG. 1B).

### Example 3

18 single nucleotide polymorphisms (SNPs) were genotyped in the MnSOD2 gene and within the group of 18, a smaller group of SNPs (shown below; Table 5) was detected at Chromosome 17 between locus rs8031 and rs5746092 with a distance 13 kb. None were in the coding region of the gene. Six tagging SNPs (Table 5) capturing most of the haplotypes in this region were selected for further statistical analysis (with mean r-square of 0.953). rs7855, the only SNP outside of the block, was also selected for further statistical analysis.

TABLE 5

| Tagging | |
|---|---|
| SNPs | Alleles Captured (with mean r-square of 0.953) |
| rs2758332 | rs2855116, rs2758331, rs2842960, rs2758339, rs5746112, |
| | rs2842959, rs2758332, rs8031, rs4880, rs2070994 |
| rs4523113 | rs5746092, rs5746094, rs4523113 |
| rs5746136 | rs5746136 |
| rs2758330 | rs2758330 |
| rs5746138 | rs5746138 |
| rs2842958 | rs2842958 |
| rs7855 | rs7855 |

For the selected 7 SNPs in MnSOD2, there was little overall association between genotypes and patient characteristics at diagnosis, including age, PSA, T stage, biopsy Gleason score, percentage of positive core at biopsy and D'Amico risk group at diagnosis (p>0.05). In this cohort, plasma selenium level was available for 499 patients. Overall, no association was found between genotypes and selenium levels (p>0.05), between selenium levels and patient baseline characteristics.

However, in 3 of 7 SNPs (rs2758330, rs2758332 and rs4523113; see Table 6), the associations of disease risk at diagnosis with selenium levels were significantly modified by the genotypes (P-values for interaction<0.05 Table 7).

TABLE 6
The distributions of genotypes for the 3 polymorphisms

| Locus | Genotype | Count | Frequency |
|---|---|---|---|
| rs2758330 | G/G | 33 | 0.04 |
| | G/T | 236 | 0.31 |
| | T/T | 488 | 0.64 |
| rs2758332 | A/A | 184 | 0.25 |
| | A/C | 363 | 0.49 |
| | C/C | 191 | 0.26 |
| rs4523113 | A/A | 38 | 0.05 |
| | A/T | 280 | 0.37 |
| | T/T | 435 | 0.58 |

For example, among patients with GG/GT genotype at rs2758330, higher selenium levels were associated with an increased chance of developing more aggressive disease (intermediate/high risk) at diagnosis (Table 7), whereas this relationship was not apparent among men with the TT genotype at rs2758330. The risk assessment patterns were similar for the associations of biopsy Gleason scores with selenium levels (Table 8).

TABLE 7
Percent of patients with intermediate/high risk disease at diagnosis by
plasma selenium levels, separately for variant genotyping statuses.

| | Quintile of plasma selenium level | | | | | |
|---|---|---|---|---|---|---|
| | I(low) | II | III | IV | V(high) | |
| | % of patients with | | | | | P-value |
| | intermediate/high risk | | | | | (interaction) |
| rs2758330 | | | | | | |
| GG and GT | 24 | 30 | 42 | 51 | 63 | 0.030* (0.044**) |
| TT | 51 | 43 | 43 | 39 | 49 | |
| rs2758332 | | | | | | |
| AA | 54 | 53 | 39 | 40 | 33 | 0.021 (0.012) |
| AC | 38 | 30 | 44 | 50 | 64 | |
| CC | 19 | 39 | 45 | 38 | 60 | |
| rs4523113 | | | | | | |
| AA and AT | 29 | 36 | 49 | 41 | 66 | 0.123 (0.044) |
| TT | 45 | 41 | 40 | 45 | 46 | |

*from Logistic regression where selenium was coded as quintile levels;
**from Logistic regression where selenium was coded as continuous values.

TABLE 8
Percent of patients with high Gleason Score at diagnosis by
plasma selenium levels, separately for variant genotyping statuses.

| | Quintile of plasma selenium level | | | | | |
|---|---|---|---|---|---|---|
| | I(low) | II | III | IV | V(high) | P-value |
| | % of patients with high Gleason (≧7) | | | | | (interaction) |
| rs2758330 | | | | | | |
| GG and GT | 17 | 27 | 36 | 44 | 50 | 0.036 (0.094) |
| TT | 46 | 31 | 38 | 33 | 39 | |
| rs2758332 | | | | | | |
| AA | 46 | 40 | 33 | 36 | 27 | 0.102 (0.102) |
| AC | 33 | 20 | 39 | 41 | 49 | |
| CC | 15 | 30 | 35 | 35 | 53 | |
| rs4523113 | | | | | | |
| AA and AT | 26 | 21 | 43 | 35 | 53 | 0.154 (0.060) |
| TT | 38 | 34 | 34 | 39 | 36 | |

In another example, among patients with AC or CC genotype at rs2758332, higher selenium levels were associated with an increased chance of developing more aggressive disease (intermediate/high risk) at diagnosis (Table 7). The risk assessment patterns were also similar for the associations of biopsy Gleason scores with selenium levels (Table 8).

For example, among patients with AA and AT genotype at rs4523113, higher selenium levels were associated with an increased chance of developing more aggressive disease (intermediate/high risk) at diagnosis (Table 7), whereas this relationship was not apparent among men with the TT genotype at rs4523113. The risk assessment patterns were similar for the associations of biopsy Gleason scores with selenium levels (Table 8).

There were very few patients with high T stage (T2b/T3/T4) at diagnosis in this cohort.

TABLE 9
Pairwise Linkage Disequilibrium (LD) measures (D′ and r²) between
the 3 polymorphisms

| | D′ (95% CI) | r² |
|---|---|---|
| rs2758330 & rs2758332 | 1.00 (0.97-1.00) | 0.245 |
| rs2758330 & s4523113 | 1.00 (0.90-1.00) | 0.077 |
| rs2758332 & rs4523113 | 1.00 (0.97-1.00) | 0.307 |

The 3 SNPs captured the majority of information in this region. They represent 14 SNPs with r-square>0.95 (see the above tagging SNPs selection).

The inverse association of men with TT genotype in rs4880 and high selenium level (4^{th} versus 1^{st} quartile) was associated with a low relative risk for total prostate cancer as well as aggressive prostate cancer was confirmed in the instant study (Table 10 and FIG. 1A; “CC”). In addition, these data indicated that there was a positive relation between selenium levels and disease risk in men with TT/TC genotype (p-interaction=0.048).

TABLE 10
Percent of patients with intermediate/high risk disease at
diagnosis by plasma selenium levels, separately for variant
genotyping statuses at rs4880.

| | Quintile levels of plasma selenium | | | | | |
|---|---|---|---|---|---|---|
| | I(low) | II | III | IV | V(high) | P-value |
| rs4880 | % of patients with intermediate/high risk | | | | | (interaction) |
| TT | 23 | 36 | 45 | 40 | 57 | 0.048 (0.028) |
| TC | 41 | 33 | 45 | 49 | 65 | |
| CC | 54 | 52 | 35 | 38 | 32 | |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining whether a man has, or is at an increased risk of developing, an aggressive form of prostate cancer, the method comprising:
providing a man having an elevated level of an antioxidant in a biological sample obtained from the man; and
determining the MnSOD2 genotype of the man, wherein the genotype is selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T), and
wherein an MnSOD2 genotype of rs4880 (T/C), rs4880 (T/T), rs2758330 (G/G), rs2758330 (G/T), rs2758332 (A/C), rs2758332 (C/C), rs4523113 (A/A), or rs4523113 (T/T) indicates that the man has, or is at an increased risk of developing, an aggressive form of prostate cancer.

2. The method of claim 1, further comprising determining that the man has an elevated level of the antioxidant in the biological sample obtained from the man.

3. A method for determining whether a man has, or is at an increased risk of developing, an aggressive form of prostate cancer, the method comprising:
providing a man having an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T); and
measuring the level of an antioxidant in a biological sample obtained from the man,
wherein an elevated level of the antioxidant in the biological sample as compared to a control level indicates that the man has, or is at an increased risk of developing, an aggressive form of prostate cancer.

4. The method of claim 3, further comprising determining the MnSOD2 genotype of the man.

5. The method of claim 1, wherein the man has been identified as having prostate cancer.

6. The method of claim 1, wherein the antioxidant comprises selenium.

7. The method of claim 1, wherein the antioxidant is selected from the group consisting of lycopene, α-tocopherol, α-carotene, β-carotene, β-cryptoxanthin, γ-tocopherol, lutein, resveratrol, and retinol.

8. The method of claim 1, wherein the antioxidant is a flavenoid.

9. The method of claim 1, wherein the MnSOD2 genotype is rs4880 (T/C).

10. The method of claim 1, wherein the MnSOD2 genotype is rs4880 (T/T).

11. The method of claim 1, wherein the MnSOD2 genotype is rs2758330 (G/G), rs2758330 (G/T), rs2758332 (A/C), rs2758332 (C/C), rs4523113 (A/A), or rs4523113 (T/T).

12. The method of claim 1, wherein the determining consists of determining whether the man has an MnSOD2 genotype of rs4880 (T/C) or rs4880 (T/T).

13. The method of claim 1, wherein the determining consists of determining whether the man has an MnSOD2 genotype of rs2758330 (G/G), rs2758330 (G/T), rs2758332 (A/C), rs2758332 (C/C), rs4523113 (A/A), or rs4523113 (T/T).

14. The method of claim 1, wherein the level of the antioxidant in the biological sample is at least 1.5 fold greater than the control level.

15. The method of claim 1, wherein the level of the antioxidant in the biological sample is at least 2 fold greater than the control level.

16. The method of claim 1, wherein the biological sample is a blood sample.

17. The method of claim 1, wherein determining the MnSOD2 genotype comprises a polymerase chain reaction (PCR).

18. The method of claim 1, wherein determining the MnSOD2 genotype comprises nucleic acid hybridization.

19. The method of claim 2, wherein measuring the antioxidant levels in the biological sample comprises inductively-coupled plasma mass spectrometry (ICP-MS).

20. The method of claim 1, further comprising obtaining the biological sample from the man.

21. The method of claim 1, further comprising detecting the presence or amount of one or both of prostate serum antigen (PSA) and prostate specific membrane antigen (PSMA) levels in a biological sample obtained from the man.

22. The method of claim 1, further comprising, if the man is determined to have, or be at an increased risk for developing, an aggressive form of prostate cancer, creating a record indicating that the subject has, or is at an increased risk of developing an aggressive form of prostate cancer.

23. The method of claim 22, wherein the record is created on a computer-readable medium.

24. The method of claim 1, further comprising, if the man is determined to have, or be at an increased risk for developing, an aggressive form of prostate cancer, prescribing an anti-cancer therapy for the man.

25. The method of claim 1, further comprising, if the man is determined to have, or be at an increased risk for developing, an aggressive form of prostate cancer, administering an anti-cancer therapy to the man.

26. The method of claim 26, wherein the anti-cancer therapy does not comprise an antioxidant.

27. The method of claim 25, wherein the anti-cancer therapy does not comprise selenium.

28. The method of claim 25, wherein the anti-cancer therapy comprises one or more of a chemotherapeutic agent, a form of radiation treatment, or an immunotherapeutic agent.

29. A method for assessing whether antioxidant therapy should be discontinued, the method comprising discontinuing an anti-cancer antioxidant therapy for a man having prostate cancer and undergoing the therapy if the man is identified as having: (i) an elevated level of the antioxidant in a biological sample obtained from the man and (ii) an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T).

30. The method of claim 29, further comprising determining the MnSOD2 genotype of the man.

31. The method of claim 29, further comprising determining that the level of the antioxidant is elevated in the biological sample.

32. The method of claim 29, further comprising administering to the subject an anti-cancer therapy that does not comprise an antioxidant.

33. A method for predicting whether a man has, or is at an increased risk of developing, an aggressive form of prostate cancer, the method comprising:
measuring the level of an antioxidant in a biological sample obtained from a man;
determining the MnSOD2 genotype of the man; and
predicting that the man has, or is at an increased risk of developing, an aggressive form of prostate cancer if (i) the level of the antioxidant in the biological sample is elevated as compared to a control level and (ii) the man has an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T).

34. A method for determining whether a man has, or is at an increased risk of developing, an aggressive form of prostate cancer, the method comprising:
providing a medical profile of a man, the profile comprising information on the level of an antioxidant in a biological sample obtained from the man and the MnSOD2 genotype of the man; and
determining whether the man has, or is at an increased risk of developing, an aggressive form of prostate cancer using the information on the level of the antioxidant and the MnSOD2 genotype,
wherein (i) an elevated level of the antioxidant in the biological sample as compared to a control level and (ii) an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T) indicates that the man has, or is at an increased risk of developing, an aggressive form of prostate cancer.

35. The method of claim 34, further comprising generating the medical profile of the man, wherein the generating comprises:
measuring the level of the antioxidant in the biological sample; and
determining the MnSOD2 genotype of the man.

36. A computer-based method for determining whether a man has, or is at an increased risk of developing, an aggressive form of prostate cancer, the method comprising:
providing the level of an antioxidant in a biological sample from a man and the MnSOD2 genotype of the man;
inputting the level of the antioxidant and the MnSOD2 genotype into a computer; and
calculating a parameter indicating whether the man has, or is at an increased risk of developing, an aggressive form of prostate cancer using the computer and the input level of the antioxidant and the MnSOD2 genotype, wherein the parameter evaluates (i) an elevated level of the antioxidant in the biological sample as compared to a control level and (ii) an MnSOD genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T) as an indication that the man has, or is at an increased risk of developing, an aggressive form of prostate cancer.

37. The method of claim 34, further comprising outputting the parameter from the computer.

38. A method for prescribing an anti-cancer therapy, the method comprising prescribing for a man in need thereof an anti-cancer therapy that does not comprise an antioxidant, wherein the man has been identified as having: (i) prostate cancer; (ii) an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T); and (iii) an elevated level of the antioxidant in a biological sample obtained from the man as compared to a control level.

39. A method for treating prostate cancer, the method comprising administering to a man in need thereof an effective amount of an anti-cancer therapy that does not comprise an antioxidant, wherein the man has been identified as having: (i) prostate cancer; (ii) an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T); and (iii) an elevated level of the antioxidant in a biological sample obtained from the man as compared to a control level.

40. A method for determining whether a man has, or is at an increased risk of developing, an aggressive form of prostate cancer, the method comprising:
measuring the level of an antioxidant in a biological sample obtained from a man,
wherein (i) an elevated level of the antioxidant in the biological sample, as compared to a control level, indicates that the man has, or is at an increased risk of developing, an aggressive form of prostate cancer.

41. The method of claim 40, wherein the man has been identified as having prostate cancer.

42. The method of claim 40, further comprising determining the MnSOD2 genotype of the man.

43. The method of claim 40, wherein the antioxidant comprises selenium.

44. An article of manufacture comprising:
a container; and
a composition contained within the container,
wherein the composition comprises an active agent for treating a prostate cancer in a man, wherein the active agent in the composition does not comprise an antioxidant, and wherein the container has a label indicating that the composition is for use in treating prostate cancer in a man if the subject has been identified as having: (i) prostate cancer; (ii) an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T); and (iii) an elevated level of the antioxidant in a biological sample obtained from the man as compared to a control level.

45. The article of claim 44, further comprising instructions for administering the composition to a man.

46. A kit for use in determining whether a man is at an increased risk of developing an aggressive form of prostate cancer, the kit comprising:
one or more reagents for determining the MnSOD2 genotype of a man; and
one or more reagents for measuring the level of an antioxidant in a biological sample obtained from a man.

47. The kit of claim 46, further comprising instructions for determining whether a man is at an increased risk of developing an aggressive form of prostate cancer.

48. The kit of claim 46, further comprising means for obtaining a biological sample from a man.

49. The kit of claim 46, further comprising a control sample containing a known amount of one or more antioxidants.

50. The kit of claim 46, wherein at least one of the one or more reagents for determining the MnSOD2 genotype of a man is a nucleic acid primer.

51. The kit of claim 46, wherein each of the one or more reagents for determining the MnSOD2 rs4880 genotype of a man comprise at least two polynucleotides that selectively hybridize to one or more of a nucleic acid sequence comprising: rs4880 (C/C), rs4880 (T/C), rs4880 (T/T), rs2758330 (G/G), rs2758330 (G/T), rs2758332 (A/C), rs2758332 (C/C), rs4523113 (A/A), or rs4523113 (T/T).

52. The kit of claim 51, wherein the probes are bound to a solid support.

53. The kit of claim 46, wherein at least one of the one or more reagents for measuring the level of an antioxidant in a biological sample obtained from a man is useful for measuring selenium levels in a biological sample.

54. A method for reducing superoxide levels in a man, the method comprising:
providing a man identified as having (i) an elevated level of an antioxidant in a biological sample obtained from the male subject and (ii) an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T); and
administering to the man a compound in an amount effective to stimulate the activity of MnSOD2 to thereby reduce superoxide levels in the man.

55. A method for reducing superoxide levels in a man, the method comprising:
providing a man identified as (i) having an elevated level of an antioxidant in a biological sample obtained from the male subject and (ii) identified as having an MnSOD2 genotype selected from the group consisting of rs4880 (T/C), rs4880 (T/T), rs2758330 (G/G), rs2758330 (G/T), rs2758332 (A/C), rs2758332 (C/C), rs4523113 (A/A), and rs4523113 (T/T); and
administering to the man a compound in an amount effective to increase the amount of MnSOD2 in the mitochondria of a cell to thereby reduce superoxide levels in the man.

56. The method of claim 54, further comprising determining that the man has an elevated level of an antioxidant in a biological sample obtained from the man.

57. The method of claim 54, further comprising determining the MnSOD2 genotype of the man.

58. The method of claim 57, wherein the compound increases the expression of MnSOD2 to thereby stimulate activity.

59. The method of claim 54, wherein the compound comprises a nucleic acid encoding MnSOD2 or a biologically active fragment thereof.

60. The method of claim 59, wherein the nucleic acid encodes a non-disease-associated form of the MnSOD2 protein or biologically active fragment thereof.

61. The method of claim 59, wherein the nucleic acid comprises a sequence encoding a mitochondrial targeting signal.

62. The method of claim 54, further comprising predicting whether the man is at an increased risk for developing an aggressive form of prostate cancer.

63. The method of claim 54, further comprising administering to the man an anti-cancer therapy.
